(19) United States
(12) Patent Application Publication
(10) Pub. No.: US 2012/0232166 A1
(43) Pub. Date: Sep. 13, 2012

(54) Masking Bitter Flavors

(76) Inventors: John W. Finley, Baton Rouge, LA (US); Darryl Holliday, Baton Rouge, LA (US); Joan M. King, Baton Rouge, LA (US); Alfredo D. Prudente, JR., Baton Rouge, LA (US)

(21) Appl. No.: 13/501,317

(22) PCT Filed: Oct. 4, 2010
(86) PCT No.: PCT/US2010/051299

§ 371 Date: Jun. 5, 2012

Related U.S. Application Data
(60) Provisional application No. 61/250,685, filed on Oct. 12, 2009.
(60) Provisional application No. 61/323,481, filed on Apr. 13, 2010.

(51) Int. Cl. A23L 1/09; A23L 1/30; A23L 2/02; A23K 1/14; A23K 1/00; A61K 47/28; A23L 1/212; A23L 1/31; A23L 1/305; A23L 1/48; A23L 1/237; A23L 2/00; A23L 1/28

(52) U.S. Cl. 514/777; 426/590; 426/658; 426/74; 426/599; 426/635; 426/615; 426/655; 426/641; 426/656; 426/592; 426/649

(57) ABSTRACT

Mogrosides may be used as a flavor modifier in various foods, beverages, pharmaceutical compositions, and the like to block the perception of bitter and astringent flavors or other off flavors.

The benefit of the 12 Oct. 2009 filing date of U.S. provisional patent application Ser. No. 61/250,685, and of the 13 Apr. 2010 filing date of U.S. provisional patent application Ser. No. 61/323,481 are claimed under 35 U.S.C. §119(e) in the United States, and are claimed under applicable treaties and conventions in all countries.

### TECHNICAL FIELD

This invention pertains to the masking of bitter flavors and other off flavors in foods, beverages, pharmaceutical compositions, and the like.

### BACKGROUND ART

Mogrosides are found naturally in the Lo Han Gou fruit (Siraitia grosvenorii Swingle). Mogrosides are used as potent sweeteners. Their use as sweeteners has been complicated by the fact that the fruit also contains bitter components, such as mogroside II. Some prior work has been directed to removing the bitter components of Lo Han Gou extract, to improve its palatability.

On 15 Jan. 2010 the United States Food and Drug Administration issued “Agency Response Letter GRAS Notice No. GRN 000301,” concluding that “the agency has no questions at this time regarding” a private applicant's “conclusion that [Siraitia grosvenorii fruit extract (SGFE)] is GRAS [generally recognized as safe] under the intended conditions of use.” See tinyurl.com/yeakvxb. To the present inventors' knowledge, no adverse effects have been reported from the use of Lo Han Guo, nor of Lo Han Guo extract, nor of mogrosides, even at concentrations substantially higher than those contemplated by the present invention.

Chang et al., WO 2008/112991 discloses the use of Lo Han Guo compositions including mogroside V to reduce the lingering bitter taste in a beverage sweetened with steviol glycoside.

May et al., WO 2008/112961 discloses beverages containing a nonnutritive sweetener, and glycerine in an amount sufficient to enhance mouthfeel and in some cases to add perceptible sweetness as well. Lo Han Guo powder is mentioned as one of several possibilities in a list of potential sweeteners.

Ungureanu et al., WO 2008/119196 discloses compounds that block “off notes” in consumables, including off notes imparted by artificial sweeteners. Mogroside V is mentioned as one of several possible artificial sweeteners in a list of potential ingredients. The mogroside itself, however, was said to impart an “off note.” It was also stated that the disclosed off-note blockers might suppress the bitterness associated with potassium. See also Givaudan et al., WO 2009/015504.

Zhou, U.S. Pat. No. 6,319,523 discloses a composition for inhibiting oral bacteria, comprising a polyphenol derivative, a mogroside derivative, and licorice extract. Each of the three components was said to have an antibacterial effect. In addition, the mogroside was said to “overcome the bitter taste of the polyphenol derivative thereby providing the composition with a more pleasant taste.” See Col. 3, lines 48-50.

McArdle et al., U.S. patent application pub. no. 20090162498 discloses that a non-sweetening amount of a potent sweetener can modify the taste of comestible grain products, changing taste characteristics such as flavor perception, sweetness perception, grassy flavor perception, bitter flavor perception, grainy flavor perception, or salty flavor perception. Mogroside V is mentioned as one of several possibilities in a list of potential sweeteners.

Bell et al., U.S patent application pub. no. 20090162484 discloses beverages containing a non-sweetening amount of a potent natural sweetener, said to modify the taste of a beverage, for example, modifying flavor impact, sweetness, bitterness, sourness, off-flavor, oiliness, sliminess, cling, or chalkiness. Mogroside V is mentioned as one of several possibilities in a list of potential sweeteners.

Frerot et al., WO 2008/129457 discloses a method for preparing a sweet juice extract from plants of the Cucurbitaceae family, including luo han guo extracts containing mogrosides.

West et al., U.S patent application pub. no. 20090196966 discloses a method for preparing a sweetener extract from plants of the Cucurbitaceae family, with objectionable flavors removed, including luo han guo extracts containing mogrosides. See also Fischer et al., U.S. Pat. No. 5,433,965; and Shi et al., U.S. Pat. No. 7,575,772.

### DISCLOSURE OF THE INVENTION

We have discovered that mogrosides may be used as a flavor modifier in various foods, beverages, pharmaceutical compositions, and the like to block the perception of bitter and astringent flavors or other off flavors.

The structures of Mogrosides III, IV, and V, and siamenoside I are shown below:

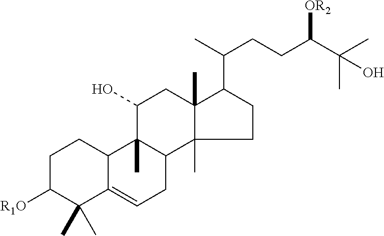

| wherein: | R₁ | R₂ |
|---|---|---|
| Siamenoside I | β-glc | β-glc²-β-glc⁶\β-glc |
| Mogroside III | β-glc | β-glc²-β-glc |
| Mogroside IV | β-glc⁶-β-glc | β-glc²-β-glc |
| Mogroside V | β-glc⁶-β-glc | 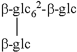 |

glc = glucopyranose

### MODES FOR PRACTICING THE INVENTION

### EXAMPLE 1

### Masking of Bitter Flavor from Potassium and Other Salts in Foods

Potassium salts such as potassium chloride create a bitter flavor in foods, limiting industry's ability to use potassium salts in low-sodium products. Sodium chloride is an excellent preservative and flavor enhancer in a variety of food and beverage applications. However, excess consumption of sodium can cause excessive water retention and hypertension. It is expected that in the future there will be increased pressure on the food industry to reduce sodium concentrations in foods. Potassium is an excellent substitute for sodium in many functions. However, its bitter flavor has previously limited its use in foods.

To demonstrate the bitter-blocking capacity of mogroside mixtures, 2% aqueous solutions of potassium chloride were prepared, either with or without 250 ppm mogroside. When tasted by volunteers, the solution without mogroside was bitter, unpleasant, and generally considered undrinkable. In marked contrast, the solution with 2% potassium chloride and mogroside was perceived as being slightly salty, otherwise bland, and far more drinkable.

Mogrosides are known in the art as natural sweeteners. When used in the present invention, however, mogrosides will block bitter flavors at concentrations much lower than one would used to impart any significant degree of sweetness, e.g., 1000 ppm, 500 ppm, 200 ppm, 100 ppm, 50 ppm, 25 ppm, or even lower. In many applications, a preferred range is 200-1000 ppm mogroside, more preferably 300-800 ppm. (Throughout the specification and claims, “ppm” means “parts per million,” as measured by mass or weight.)

Mogroside can mask the bitter note and, to some extent, the salty note associated not just with potassium salts, but also with other salts, such as those of calcium, sodium, or magnesium.

### EXAMPLE 2

### Masking of Biting Flavor from Glycerine in Foods

Glycerine causes an astringent or biting taste in the back of the mouth when added to foods at levels over a few percent by weight. We have found that adding mogroside to glycerine-water mixtures greatly reduces or even eliminates the astringent or biting taste. Glycerine may then be used as a humectant in intermediate-moisture human and pet foods. Adding a preferred concentration of about 200 ppm mogroside reduces or even eliminates the glycerine “bite.” Foods such as beans, pasta, rice, and bulgur can be pre-cooked in aqueous solutions of glycerine (˜2 to ˜30% by weight), containing ˜25 to ˜500 ppm mogroside. The products are then optionally dried to a water activity (aw) less than ˜0.7. The resulting materials can be rehydrated quickly with no bitter flavor.

Beans such as black beans, navy beans, pinto beans, and other beans can be cooked and dehydrated in a similar manner. The beans can be rehydrated rapidly with no off flavors. Alternatively, the cooked and dehydrated beans may be used as snack products with textures similar to peanuts.

### EXAMPLES 3-8

### Masking of Bitter Flavors and Off-Flavors in Foods, Medical Foods, Vegetables, Other Foods, and Pharmaceutical Compositions

Mogroside may also be used to improve the flavors of, or reduce the bitterness, astringency, or off-flavors in baked products, in vegetables, organ meats, game, other foods, acai purees, acai juice, other acai products, medical foods, pharmaceutical products, alcoholic beverages, and the like. Mogroside may be used to smooth out harsh flavors in lower-cost beverages, such as Robusta coffee, muscadine juice, or fusel oil flavors in distilled spirits.

Acai berries have a bitter off-flavor, which has limited the use of acai as a natural anti-oxidant. Prior methods for minimizing the bitterness of acai have included diluting the juice, adding sugar, or mixing acai with other, sweeter berries. We have found that when mogroside is added to acai products, such as acai berry puree, it masked the bitterness and off-flavor notes of the acai berry ingredients, so that acai may be used at higher concentrations without the undesirable off-flavors or bitterness. The flat, tannic notes of acai were masked, and the puree flavor changed to a semi-sweet berry flavor with large, juicy top notes. The preferred concentration range was ˜200 to ˜500 ppm mogroside. At 800 ppm, a strong, high-intensity sweet flavor was obtained. Possible uses include any products that contain acai berry ingredients, including for example raw berries, purees, powders, flavorings, juices, ice cream (with or without glycerin), fruit fillings, smoothies, yogurts, teas, coffee, beer, salad dressing, marinades, candies, wines, liquors, other alcoholic beverages, and the like.

Possible uses in other food products include foods that otherwise have a bitter or off-flavor, including for example raw berries, purees, powders, flavorings, juices, ice cream (with or without glycerin), fruit fillings, smoothies, yogurts, vegetables, teas, coffee, beer, salad dressing, marinades, candies, wines, liquors, and other alcoholic beverages. In many applications (whether foods, beverages, or pharmaceutical compositions), a preferred range for mogroside is 200-1000 ppm mogroside, more preferably 300-800 ppm. Optimal ranges for use in a particular application may readily be determined through routine testing.

Mogroside may also be applied to vegetables, for example as a spray or as a dipping solution. The bitter and astringent flavor notes of many vegetables are thereby reduced substantially, resulting in a smoother-tasting vegetable. Mogroside can also reduce beany notes and off-flavors in vegetables. We dispersed an aqueous or ethanol solution of mogroside into vegetable oil at a concentration of 800 ppm. The vegetable oil was then sprayed onto vegetables, either before or after cooking. With lima beans we observed a reduction in bitterness. In artichoke hearts we observed a reduction in bitterness and grassy flavor. In peas we observed a shorter flavor profile. In broccoli we observed a reduction in sulfur and bitter notes. In green beans we observed a sweeter flavor and fewer beany notes. In carrots we observed a more intense sweetness. In corn we observed a sweeter corn flavor. We observed that the vegetable oil spray worked well, and that a topical spray after cooking seemed to be more effective than application before cooking. The mogroside may be applied to vegetables from aerosols or misting solutions. It may also be used in microwaving, steaming, or boiling of vegetables, as well as other methods of cooking. The changes in the bitterness and off-flavors of vegetables could help improve their palatability for children or adults who do not otherwise like vegetables. Many people simply do not like vegetables, despite their well-publicized health benefits. Treating vegetables with mogroside can help to improve their acceptability.

Mogroside may be used in foods and beverages containing polyphenolic antioxidants, other polyphenolic compounds, and other phytochemicals to make more palatable products, while delivering significant potential health benefits. For example, mogroside can help reduce the bitterness attributed to polyphenolics and other botanical flavors in cocoa, acai, other berries, tea, beer, grapes, wine, olive oil, other vegetable oils, chocolate, cocoa, coffee, walnuts, other nuts, peanuts, borojo, pomegranates, popcorn, yerba mate, and other fruits and vegetables.

Free amino acid-based medical foods are indicated for patients with certain conditions, for example cancer patients who are unable to eat solid protein foods, patients with compromised digestive systems, and patients taking certain weight loss formulations. Amino acid formulations lacking methionine can help slow cancer growth. Unfortunately, free amino acids are notoriously bitter and unpleasant-tasting, and it can be difficult to maintain patient compliance. Previous flavoring formulations have added sweetness and other flavors, but have not successfully masked the bitterness.

We have found that mogroside can help reduce the bitterness, unpleasant mouthfeel, and off-flavors associated with amino acid-based medical foods, other medical foods, other amino acid-fortified foods, nutritional supplements, protein hydrosylates, and pharmaceuticals. For example, we added mogroside to a commercial amino acid beverage (Hominex™) at a rate of 200-1000 ppm, preferably 300-800 ppm. The Hominex™ suspension, which contains free amino acids, vitamins, minerals, and other nutrients, was prepared in accordance with the manufacturer's directions. Mogroside was then added to the dissolved medical food drink. The suspension was allowed to equilibrate for about one minute before tasting. Compared to the unmodified beverage, there was a marked reduction in the off-flavors, the chalkiness, the pastiness, and the bitterness. These attributes were reduced at 200 ppm mogroside, and were essentially eliminated with 300 ppm mogroside. At around 600 ppm a mogroside-associated off flavor began to develop, which became pronounced at 800 ppm. Unless additional flavors are also added, mogroside's own off-flavor can limit its use at higher concentrations. Conversely, at least for the samples we have tested to date, there may be no compelling reason to use mogroside at such higher levels anyway. Using mogroside in free amino acid-based foods can reduce or eliminate the bitter and unpleasant flavors, making them much more palatable, and enhancing patient compliance. Similar results are expected for improving the palatability of other medical foods.

Mogroside may be used to improve the flavor of many pharmaceutical compositions that are taken orally, and thereby to improve patient compliance. For example, many pediatric pharmaceutical compositions are taken orally as suspensions or syrups. The typical childhood opinion about the taste of many a medicine (e.g., an antibiotic) is too well known to require citation. Adding mogroside to these oral pharmaceutical compositions will improve their acceptability. Another example is the polyethylene glycol/electrolyte solution that is often taken by a patient to cleanse the bowel before a colonoscopy or other intestinal procedure. A relatively large volume of a relatively unpleasant-tasting liquid must be taken orally. Various flavorings have previously been used to partially mitigate the unpleasant taste, but there remains much room for improvement. Mogroside will help reduce the bitterness and saltiness of these preparations, and thereby improve patient compliance.

### EXAMPLE 9

### Masking of Bitter Flavors and Off-Flavors in Liver and Other Organ Meats

Mogrosides may also be used to reduce the bitter flavors and off-flavors in liver and other organ meats, or in game meat. Some individuals have a strong aversion to eating organ meats, especially liver, due to the bitter flavors and off-flavors that they often present. Mogrosides can help to mask these bitter and off flavors, making liver and other organ meats more palatable.

In initial experiments, Mogroside V was tested at varying levels from 100-500 ppm. Preferred levels of mogroside were found to be between about 150 ppm and about 250 ppm, most preferably about 200 ppm. Below the preferred range, the effect was minimal; and above the preferred range, a very sweet taste developed. Mogroside V was added to a chicken liver and chicken gizzard puree, and the puree was poached to an internal temperature of 74° C. A control puree with no mogroside was identically cooked. Six individuals tasted the samples blind and could easily differentiate them. The control was described as “livery,” “organy,” or “bloody”; while the test sample at the 150-250 ppm range was deemed slightly sweet, “bologna-like,” and “bland.” All testers preferred the samples with mogroside, and the majority preferred a level of 200 ppm.

In another experiment, mogroside was added to a beef liver puree, and the puree was poached to an internal temperature of 74° C. A control puree with no mogroside was identically cooked. Six individuals tasted the samples blind and could easily differentiate them. The control was again described as “livery,” “organy,” or “bloody”; while the test sample at the 150-250 ppm range was deemed slightly sweet, “bologna-like,” and “bland.” All testers preferred the samples with mogroside, and the majority preferred a level of 200 ppm.

Addition of mogroside will also help improve the flavor of other organ meats and products containing organ meats. Other methods of cooking may also be used. The concentration or amount of mogroside may vary, depending on the method of cooking used. (Differential effects of different types of cooking are not limited to organ meats, but will also apply to other uses of mogroside as well.) For example, when cooking liver or other foods in oil, mogroside tends to partition from the oil into the water in the liver tissue, because the solubility of mogroside in water is much higher than that in oil. By contrast, cooking foods in water may require that higher concentrations of mogroside be used than when cooking in oil, because the mogroside is soluble in the cooking liquid. Higher concentrations of mogroside may be needed to promote osmosis or diffusion into the food. Temperature and cooking time will also have an effect on uptake of mogroside: In general, more mogroside will be taken up by food when the cooking temperature is higher, and also when the cooking time is longer.

In many (but not necessarily all) uses of the present invention, it is preferred to use levels of mogroside below the threshold at which the mogroside imparts a sweetening effect. This result may be achieved by selection of the particular mogroside or mogrosides used, or the concentration, or both. Mogroside V is sweet. Mogroside III has been reported to be tasteless. Our preliminary tests indicate that Mogroside III also effectively blocks bitter tastes. If desired, it should be possible to use Mogroside III in higher concentration than Mogroside V to block bitter or other unwanted tastes, without imparting any sweetening effect.

### EXAMPLE 10

### Mogroside V Purification Procedure

We used the following procedure to prepare gram quantities of decolorized, crystalline, Mogroside V (Mog V), CAS NUMBER [88901-36-4]:

Dissolve a raw, powered luo han guo mogroside extract, purchased from a commercial source, in water (approx 2 g/10 ml).

Pass the dissolved sample through a column of Amberlite™ IRA-410 basic anion exchange resin in chloride form (200 g) with 250 ml water.

Purify by isocratic elution on silica gel (200 g) with chloroform-methanol-water (45:12:2), using many column volumes, to obtain approx. 30-40 fractions, which are inspected by TLC for the presence or absence of Mogroside V. The melting point is ˜194-196° C.

Alternatively, the methods described in the literature cited in the background section above may be used to prepare purified mogroside V for use in the present invention. Other mogrosides, such as mogroside III, mogroside IV, and siamenoside I may alternatively be used in lieu of the preferred Mogroside V.

### EXAMPLE 11

### Mogroside III Semisynthesis and Purification

Mogroside III typically occurs early in the development of plants. In more mature plants Mogroside III appears to be absent, or at least not present in high concentrations. Purified mogroside III is not currently available from commercial sources. We therefore developed a semisynthesis of Mogroside III, using as the starting material a commercially-obtained mogroside powder that is high in Mogroside V. The semisynthesis is based on the enzymatic hydrolysis of Mogroside V with a glucosidase, as outlined below:

Materials:
1. Mogroside powder (commercially obtained)
2. Glucosidase from Aspergillus niger (Sigma 49219)
3. n-Butanol
4. Chloroform
5. Denatured ethanol

Hydrolysis and Extraction:
1. Two grams of mogroside powder were transferred into a scintillation vial.
2. Five mL of glucosidase solution (1 EU/ml in 670 mM aqueous phosphate buffer, pH 7.1) was added to the vial and mixed by swirling.
3. The solution was incubated in a 37° C. water bath for 24 hours.
4. After incubation, the solution was placed in a boiling water bath for 15 minutes.
5. After cooling, the solution was transferred to a 500-mL separatory funnel. The vial that had held the solution was washed with 3×10 mL distilled water, and that water was combined with the solution in the separatory funnel.
6. The aqueous solution in the separatory funnel was partitioned consecutively with chloroform and n-butanol. First, 100 mL chloroform was added, the separatory funnel was shaken for 1 minute, and the chloroform was removed. Then 100 mL n-butanol was added to the aqueous layer remaining in the separatory funnel, and the funnel was again shaken for 1 minute.
7. The butanol layer was collected in an evaporating flask, and evaporated almost to dryness under vacuum.
8. The syrupy material remaining after the removal of butanol was dissolved in denatured alcohol, which caused Mogroside III to precipitate.
9. The alcohol was removed by evaporation under vacuum, without heating.
10. The precipitate was scraped and transferred into a pre-weighed vial.
11. The mass of the collected material was 0.45 grams. This material is believed to comprise primarily Mogroside III. Additional testing and characterization will be conducted to confirm the identity and purity of the collected material.

### EXAMPLES 12-31

Additional examples of the use of Mogroside V to alter the flavors of various consumables, and the experimental results obtained, are summarized in Table I:

TABLE I

| KCl | 2% | | bitter |
|---|---|---|---|
| | 2% + 250 ppm mogroside | | salty |
| | 2.5% + 250 ppm mogroside | | salty |
| | 3.0% + 250 ppm mogroside | | slight bitterness |
| Coconut water | plain | | bitter |
| | plain + 200 ppm mogroside | | sweet |
| Cocoa Powder | plain | | bitter |
| | plain + 200 ppm mogroside | | chocolate |
| Pomegranate juice | plain | | tannic |
| | plain + 200 ppm mogroside | | sweet |
| Lima beans | plain | | beany |
| | plain + 100 ppm mogroside in oil | | semi-sweet; no beaniness |
| Turnip greens | plain | | bitter |
| | plain + 100 ppm mogroside in oil | | semi-sweet; no beaniness |
| Powerade ™ sports | plain | | sweet |
| drink with | plain + 2% KCl | | bitter |
| electrolytes | plain + 2% KCl + mogroside | | salty |
| | plain + 3% KCl | | bitter |
| | plain + 3% KCl + mogroside | | slightly bitter, salty |
| Vodka | plain | | sharp bite |
| | plain + 200 ppm mogroside | | tasteless |
| Rum | plain | | sharp and bitter |
| | plain + 200 ppm mogroside | | sweet |
| Glycerine | 50% glycerine solution | | biting |
| | 50% solution + mogroside | | sweet |
| Soy Milk | plain | | beany |
| | plain + 200 ppm mogroside | | decreased beany flavor |
| Hominex ™ amino | plain + water | | bad aftertaste |
| acid formulation | plain + water + 200 ppm mogroside | | drinkable |
| (10 g Hominex ™/ | | | |
| 230 g water) | | | |
| (10 g Hominex ™/ | | | |
| 230 g water) | | | |
| Hominex ™ | plain + | | bitter, chalky, floury |
| (9.3 g Hominex ™/ | 100 ppm mogroside + | | less so, but still floury |
| 100 ml water) | 200 ppm mogroside + | | much less so, but still |
| | | | bitter & slightly floury |
| | 300 ppm mogroside + | | drinkable |
| | 400 ppm mogroside + | | tasteless |
| | 500 ppm mogroside + | | tasteless |
| | 600 ppm mogroside + | | slight strange taste |
| | 700 ppm mogroside + | | hint of off-note |
| | 800 ppm mogroside | | off-flavored |
| | Range 200-1000 ppm mogroside | | |
| | Preferred 300-800 ppm mogroside | | |
| Robusta Coffee with | plain | | bitter |
| Chicory | plain + 175 ppm mogroside | | mellow bitterness and acidity |
| 2 g/250 g water | plain + 200 ppm mogroside | | mellow with semi-sweet |
| | | | coffee flavor |
| Control | 2 Tbp. cocoa powder | | |
| | 1 c. water | | |
| | 1 tsp. non-fat dry milk | | |
| | 0.1 g salt | | |
| | 0.2 g xanthan gum | | |
| Test | Same as above + | | |
| | 400 ppm mogroside | | |
| Difference testing | Yes | No | |
| | 6 | 0 | |
| Preference testing | Control | Test | |
| | 1 | 6 | |

| | | |
|---|---|---|
| Cranberry Juice | plain | tannic, sour, puckering |
| | plain + 100 ppm mogroside | no difference from plain |
| | plain + 200 ppm mogroside | less sour, more drinkable |
| | plain + 300 ppm mogroside | slight sourness |
| | plain + 400 ppm mogroside | hint of sour, starting to taste sweet |
| | plain + 500 ppm mogroside | mildly sweet, cranberry, hint of sour |
| | plain + 600 ppm mogroside | slightly sweet, hint of sour |
| | plain + 700 ppm mogroside | faint sour, sweet, cranberry, hint of |
| | | high intensity flavor |
| | plain + 800 ppm mogroside | sweet, high intensity sweetness |
| | | flavor |
| Grapefruit Juice | plain | sour, tangy, bitter |
| | plain + 100 ppm mogroside | no difference from plain |
| | plain + 200 ppm mogroside | sour, tangy, slightly bitter |
| | plain + 300 ppm mogroside | hint of sour, hint of bitter, little |
| | | tanginess |
| | plain + 400 ppm mogroside | hint of sour, hint of bitter, very little |
| | | tangy |
| | plain + 500 ppm mogroside | no difference from 400 ppm |
| | plain + 600 ppm mogroside | almost no sourness, hint of bitter |
| | | and tanginess |
| | plain + 700 ppm mogroside | no difference from 600 ppm |
| | plain + 800 ppm mogroside | mild sweetness, high intensity flavor |
| | | on lips |
| Ruby Red | plain | mild sour, mild sweet, very mild |
| Grapefruit | | aftermath |
| | plain + 100 ppm mogroside | no difference from plain |
| | plain + 200 ppm mogroside | mild sour, sweet, high intensity |
| | | flavor |
| | plain + 300 ppm mogroside | sweet, hint of sour, aftertaste of |
| | | bitterness |
| | plain + 400 ppm mogroside | very sweet, no sour, hint of |
| | | bitterness |
| | plain + 500 ppm mogroside | very sweet, no sour, hint of |
| | | bitterness, odd last note |
| | plain + 600 ppm mogroside | too sweet |
| | plain + 700 ppm mogroside | not tested |
| | plain + 800 ppm mogroside | not tested |
| Acai | Control | bitter, tannic |
| | 100 ppm mogroside | bland, slight tannic |
| | 200 ppm mogroside | fruity, berry semi-sweet |
| | 300 ppm mogroside | tannic, little flavor at all |
| | 400 ppm mogroside | jammy, slight tannic |
| | 500 ppm mogroside | jammy, no tannin |
| | 600 ppm mogroside | jammy, sweet after taste; high |
| | | intensity flavor |
| | 700 ppm mogroside | sweet |
| | 800 ppm mogroside | too sweet |
| Acai puree | plain | tannic |
| 12% Brix | plain + 500 ppm mogroside | berry-semi-sweet/fruity |
| Acai Ice Cream | plain | hard and icy; tannic, not sweet |
| | plain + glycerine | soft and creamy, slightly grainy, |
| | | tannic, not sweet |
| | plain + glycerine + | soft and creamy, sweet, berry, mild; |
| | 300 ppm mogroside | best texture |
| Acai Ice Cream | plain + | plain, slightly gummy texture |
| | 10% Acai + | tannic, icy |
| | 5% Acai + 5% glycerin + | not much flavor, slightly softer |
| | 10% Acai + 5% glycerin + | tannic, slightly soft |
| | 10% Acai + 10% glycerin + | tannic, soft and easy to scoop, |
| | | creamy |
| | 5% Acai + 5% glycerin + | not too much flavor, slightly soft |
| | 200 ppm mogroside + | |
| | 10% Acai + 5% glycerin + | berry notes, slightly soft |
| | 200 ppm mogroside + | |
| | 10% Acai + 10% glycerin + | berry notes, easy to scoop, creamy |
| | 200 ppm mogroside + | |
| | 5% Acai + 5% glycerin + | not much flavor, slightly soft, sweet |
| | 300 ppm mogroside + | |
| | 10% Acai + 5% glycerin + | berry flavor, soft and creamy |
| | 300 ppm mogroside + | |
| | 10% Acai + 10% glycerin + | berry flavor, soft and creamy |
| | 300 ppm mogroside + | |
| | 5% Acai + 5% glycerin + | sweet, not much flavor, some berry, |
| | 400 ppm mogroside + | soft |
| | 10% Acai + 5% glycerin + | sweet, jammy, creamy, a bit too |
| | 400 ppm mogroside + | sweet |
| | 10% Acai + 10% glycerin + | sweet, jammy, creamy, a bit too |
| | 400 ppm mogroside + | sweet |
| | 300 ppm mogroside (only) | plain, gummy |

### EXAMPLES 32-39

Several sodium reduction blends were formulated and tested, as shown in Table II.

TABLE II

| | Blend Number and Formulation | | | | | | | |
|---|---|---|---|---|---|---|---|---|
| Ingredient | 1 | 2 | 3 | 4 | 5 | 6 | 7 | 8 |
| KCl (g) | 68.77 | 56.73 | 50 | 75 | 74.25 | 61.9 | 74.25 | 74.25 |
| NaCl (g) | — | 24.75 | 50 | 25 | 24.75 | 37.1 | 24.75 | 24.75 |
| CaCl2 (g) | 30.23 | 17.52 | — | — | — | — | — | — |
| Canola Oil (mg) | 500 | 500 | — | — | 500 | 500 | 500 | 500 |
| Anti-Caking Agent (mg) | 500 | 500 | — | — | 500 | 500 | 500 | 500 |
| Mogroside (mg) | 20 | 20 | — | — | 20 | 22.5 | 23.8 | 23.8 |

These sodium reduction blends were tested with red beans, navy beans, crackers, and potato chips. Results are shown in Table Ill (in which “M” is an abbreviation for Mogroside V.)

TABLE III

| | | |
|---|---|---|
| Salt Blend | Overall Taste | Saltiness |

Whole Wheat Crackers

| | | |
|---|---|---|
| NaCl | 7.04 | 6.96 |
| 69.46KCl:30.54CaCl2 | 5.50 | 5.00 |
| 68.77KCl:30.23CaCl2 + 200 ppm M | 5.83 | 5.58 |
| 57.3 KCl:25 NaCl:17.7CaCl2 | 4.92 | 5.58 |
| 56.73KCl:24.75 NaCl:17.52CaCl2 + | 5.83 | 5.58 |
| 200 ppm M | | |

Shredded Wheat Crackers

| | | |
|---|---|---|
| NaCl | 6.04 | 6.04 |
| 69.46KCl:30.543CaCl2 | 3.75 | 3.92 |
| 68.77KCl:30.23CaCl2 + 200 ppm M | 3.42 | 3.75 |
| 57.3KCl:25 NaCl:17.7CaCl2 | 3.50 | 3.92 |
| 56.73KCl:24.75 NaCl:17.52CaCl2 + | 4.92 | 5.08 |
| 200 ppm M | | |

Potato Chips

| | | |
|---|---|---|
| NaCl | 6.97 | 6.92 |
| 57.3 g KCl:25 g NaCl:17.7 g CaCl2 | 6.11 | 6.11 |
| 56.73 KCl:24.75 NaCl:17.52 CaCl2 + | 5.78 | 5.78 |
| 200 ppm M | | |
| 69.46KCl:30.54CaCl2 | 5.00 | 4.82 |
| 68.77KCl:30.23CaCl2 + 200 ppm M | 5.64 | 5.36 |

Red Beans

| | | |
|---|---|---|
| NaCl | 6.79 | 6.63 |
| 62.5KCl:37.5 NaCl | 5.83 | 5.75 |
| 61.9KCl:37.1 NaCl + 225 ppm M | 3.42 | 3.92 |
| 75KCl:25 NaCl | 5.58 | 4.75 |
| 75KCl:25 NaCl + 200 ppm M | 4.83 | 4.75 |

Navy Beans

| | | |
|---|---|---|
| NaCl | 6.17 | 6.13 |
| 75 g KCl:25 g NaCl | 4.33 | 4.67 |
| 75 g KCl:25 g NaCl + 225 ppm M | 6.25 | 6.58 |
| 62.5KCl:37.5 NaCl | 5.08 | 5.33 |
| 61.9KCl:37.1 NaCl + 225 ppm M | 3.08 | 3.83 |

Optional Compositions, Uses, and Ranges include the following:
1—A mixture containing a mogroside with from two to eight glucosides.
2—A mixture containing a mogroside with 4 to 6 glucosides.
3—A beverage containing 0.5 to 5% potassium salts, such as potassium chloride, with mogroside at a concentration of 500 ppm or lower.
4—A coating for salty snacks comprising a mixture of potassium chloride 20-80%, sodium chloride 0-50%, and mogroside 0.2 to 2%.
5—A table salt substitute comprising potassium chloride 20 to 99.8%, Sodium chloride 0 to 50%, and mogroside 0.2 to 2%.
6—A beverage containing mogroside and glycerine, without the unpleasant taste of glycerine.
7—An intermediate-moisture food containing mogroside and glycerine, without the unpleasant taste of glycerine, to replace sugar or high-fructose corn syrup.
8—An oral medicinal formulations, such as Listerine™ or Robitussin™, with added mogroside to reduce the medicinal flavor.
9—A formulation containing mogroside to block bitter notes in shelf-stable, intermediate-moisture pet foods
10—Glycerine with mogroside in solution can be used to replace some or all sugar in canned or frozen fruit products, retaining the viscosity and texture protection of sugar, but using about half as much glycerine in lieu of sugar. The result is a low-glycemic, lower-calorie product with similar texture to a sugar-syrup product. The glycerine/mogroside mixture may be used, for example, with solid pieces of fruit, fruit pastes, fruit jams, and fruit pieces in desserts. It may be used in canned or frozen products.
11—Glycerine with mogroside in solution can be used to replace some or all sugar in lower sugar pastries and dessert products, retaining the viscosity and texture protection of sugar, but using about half as much glycerine in lieu of sugar. The result is a low-glycemic, lower calorie product with similar texture to full sugar products.
12—Mogroside can be used in foods or beverages containing polyphenolic antioxidants and other phytochemicals to make a more palatable product with significant potential health benefits.
13—Mogroside can be used in foods or beverages containing other herbal supplements or phytochemicals to make a more palatable product with significant potential health benefits.
14—Mogroside can be used in foods or beverages containing high protein levels, e.g. soy protein, to make a more palatable product with significant potential health benefits.

The complete disclosures of all references cited in this specification are hereby incorporated by reference. Also incorporated by reference are the complete disclosure of the priority applications, U.S. provisional applications 61/250,685 and 61/323,481. Also incorporated by reference is the following presentation by the present inventors: D. Holliday et al., “Development of a Sodium-Free Salt for Food Applications,” Abstract, Meeting of the American Chemical Society (Boston, Mass., Aug. 27, 2010); D. Holliday et al., “The Use of Mogroside to Block Bitterness in Foods,” Slide Presentation, Meeting of the American Chemical Society (Boston, Mass., Aug. 27, 2010); In the event of an otherwise irreconcilable conflict, however, the present specification shall control.

“Palatability” and similar terms are, of course, subjective concepts to some degree. As used in the specification and claims, “palatability” and similar terms are comparative; viz., the pertinent question is whether a majority of consumers of the particular product find the palatability of the product to be improved when it contains mogroside, as compared to an otherwise substantially identical product lacking mogroside. The “consumers” should be those who would most typically consume the particular product. E.g., for an oral antibiotic suspension, typical consumers would be human children; for an oral polyethylene glycol/electrolyte solution to cleanse the bowel, typical consumers would be humans 50 years and older; for a cooked vegetable, typical consumers would be humans of all ages; for an alcoholic beverage, typical consumers would be human adults; for a pet food, typical consumers would be dogs or cats; and so forth.

What is claimed:

1. A composition of matter comprising:
(a) a consumable, wherein the consumable comprises a human or veterinary food product, a beverage, a medical food, or a pharmaceutical composition;
(b) a potassium salt; and
(c) a mogroside;
wherein:
(d) the palatability of the composition is substantially improved, as compared to the palatability of an otherwise substantially identical composition lacking mogroside.

2. A composition as in claim 1, wherein the potassium salt comprises between about 0.05% and about 5% of the consumable by mass.

3. A composition as in claim 1, wherein the potassium salt comprises between about 0.5% and about 2% of the consumable by mass.

4. A composition as in claim 1, wherein the potassium salt comprises potassium chloride.

5. A composition as in claim 1, additionally comprising a calcium salt.

6. A composition as in claim 1, wherein the mogroside comprises mogroside III.

7. A composition as in claim 1, wherein the mogroside comprises mogroside IV.

8. A composition as in claim 1, wherein the mogroside comprises mogroside V.

9. A composition as in claim 1, wherein the mogroside comprises siamenoside I.

10. A composition as in claim 1, wherein the mogroside is between about 200 ppm and about 1000 ppm by mass.

11. A composition as in claim 1, wherein the mogroside is between about 300 ppm and about 800 ppm by mass.

12. A composition of matter comprising:
(a) a consumable, wherein the consumable comprises a human or veterinary food product, a beverage, a medical food, or a pharmaceutical composition; and
(b) a mogroside;
wherein:
(c) the palatability of the composition is substantially improved, as compared to the palatability of an otherwise substantially identical composition lacking mogroside.

13. A composition as in claim 12, wherein the consumable comprises a human food product.

14. A composition as in claim 12, wherein the consumable comprises acai berry or an acai berry extract.

15. A composition as in claim 12, wherein the consumable comprises an organ meat.

16. A composition as in claim 12, wherein the consumable comprises liver.

17. A composition as in claim 12, wherein the consumable comprises gizzard.

18. A composition as in claim 12, wherein the consumable comprises a food product and glycerine.

19. A composition as in claim 12, wherein the consumable comprises a medical food.

20. A composition as in claim 12, wherein the consumable comprises a medical food product containing free amino acids.

21. A composition as in claim 12, wherein the consumable comprises a food product fortified with free amino acids.

22. A composition as in claim 12, wherein the consumable comprises a pet food.

23. A composition as in claim 12, wherein the consumable comprises a beverage.

24. A composition as in claim 12, wherein the consumable comprises an alcoholic beverage.

25. A composition as in claim 12, wherein the consumable comprises a pharmaceutical composition for oral administration.

26. A composition as in claim 12, wherein the consumable comprises a vegetable.

27. A composition as in claim 12, wherein the mogroside comprises mogroside III.

28. A composition as in claim 12, wherein the mogroside comprises mogroside IV.

29. A composition as in claim 12, wherein the mogroside comprises mogroside V.

30. A composition as in claim 12, wherein the mogroside comprises siamenoside I.

31. A composition as in claim 12, wherein the mogroside is between about 200 ppm and about 1000 ppm by mass.

32. A composition as in claim 12, wherein the mogroside is between about 300 ppm and about 800 ppm by mass.

33. A composition as in claim 12, wherein the composition comprises a calcium salt.

34. A table salt substitute comprising, by mass: potassium chloride 20% to 99.8%; sodium chloride 0% to 50%; and mogroside 0.2% to 2%.
